(19) United States
(12) Patent Application Publication
(10) Pub. No.: US 2004/0148199 A1
(43) Pub. Date: Jul. 29, 2004

(54) System for acquiring, storing, and transmitting patient medical data

(76) Inventor: Norwood P. Dixon JR., Volente, TX (US)

Correspondence Address: Michael A. O'Neil, P.C., Suite 820, 5949 Sherry Lane, Dallas, TX, 75225, US

(21) Appl. No.: 10/759,907

(22) Filed: Jan. 16, 2004

Related U.S. Application Data
(63) Continuation of application No. 09/628,780, filed on Jul. 31, 2000.
(60) Provisional application No. 60/146,717, filed on Aug. 2, 1999.

(51) Int. Cl. G06F 17/60; A61B 5/00

(52) U.S. Cl. 705/2; 705/3; 600/300

(57) ABSTRACT

A system for acquiring, storing, and transmitting patient medical data includes an apparatus supportable on the body on the patient for acquiring patient medical data. The apparatus includes a memory for storing the acquired medical data, a keyboard for inputting data to the memory, and a display for displaying data inputted to and received from the memory. A modem within the apparatus transmits patient medical data to remote locations through a preselected communication system. The apparatus further includes a GPS detector for continuously monitoring the location of the patient.

### TECHNICAL FIELD

This invention relates generally to the acquisition, storage and transmission of patient medical data, and more particularly to an apparatus worn on the wrist of the patient which acquires, stores, and transmits to remote locations patient medical data.

### BACKGROUND AND SUMMARY OF THE INVENTION

At the present time, the acquisition and storage of patient medical data is an almost entirely manual operation. Typically, the patient must visit a doctor's office and a laboratory in order that the required medical data, such as temperature, pulse, blood pressure, cholesterol levels, blood sugar level, blood thinner level, etc. can be acquired. Following acquisition, the conventional practice is to enter the data manually into a paper file.

In the case of healthy patients, the foregoing procedure is required at least annually. In the case of patients having significant illnesses, the procedure must be accomplished much more frequently, such as weekly or even daily. In the latter instances, the requirement of visiting a doctor's office and laboratory to have the necessary tests performed is time consuming to the point of presenting a major lifestyle disruption.

Automated systems for taking measurements of temperature, blood pressure, etc. are known. Such apparatus is frequently provided at pharmacies, physical training rooms, etc. Thus, it is still necessary for the patient to visit the facility at which the testing apparatus is installed in order to have such tests performed.

In the case of patients suffering from diabetes and other conditions which require blood testing, it is known to provide the patient with a testing strip which receives a droplet of the patient's blood. The strip is then inserted into an electronic reader which generates a read out of the tested condition. However, such devices do not include apparatus for transmitting the acquired data to the doctor's office of laboratory so that it can be analyzed and compared with prior data.

It is also known to connect monitoring devices to home-bound patients which are in turn connected to the doctor's office, laboratory, etc., through telephone connections, PDA's, etc. In general, such devices are non-portable in any realistic sense.

The present invention comprises a system for acquiring, storing, and transmitting patient medical data which overcomes the foregoing and other problems long since associated with the prior art. In accordance with the broader aspects of the invention, a testing apparatus is worn on the patient. The apparatus acquires medical data such as temperature, pulse, blood pressure, cholesterol levels, blood sugar level, blood thinner level, etc. from the patient, then stores the acquired data in a memory. The apparatus is then connected through an output jack to a telephone line whereupon the acquired data is transmitted to the doctor's office or to the laboratory either directly through a telephone line connection or indirectly through the Internet.

Although typically described in terms of a single patient, the apparatus has sufficient capacity to serve a group of patients, such as a family. However, in the case of a patient requiring constant monitoring, multi-patient usage would be impractical.

In accordance with more specific aspects of the invention, a patient medical data acquisition apparatus is mounted on a strap which facilitates the positioning of the apparatus on the wrist, leg, etc., of the patient. Temperature, pulse, and blood pressure sensors are mounted on the apparatus for engagement with the skin of the patient, thereby enabling the direct acquisition of patient medical data. The apparatus further includes one or more strip receiving slots which receive strips having droplets of the patient's blood applied thereto. The apparatus analyzes the blood-bearing strips to determine information such as cholesterol levels, blood sugar level, blood thinner level, etc. All of the acquired data is transferred to a memory contained within the apparatus for subsequent transmittal.

The slot(s) is designed to accept as many devices as possible. Thus, the slot is designed as a “standard” slot analogous to slots in a computer.

1 USB (Universal Serial Bus) will accept multiple devices (up to 128 devices simultaneously). All devices which now have communication capabilities can be chained to this port. Therefore, the apparatus handles all devices that communicate whether they are rare, expensive and/or less portable.

1 USB is reserved for direct connection to a computer, PDA (personal digital assistant) or other connection such as cable, etc.

The mechanical communication slot contains an RJ11/RJ14 phone

The ‘Blue Tooth’ standard function can also be included. This is a wireless port performing the same function that the USB performs.

The apparatus will include the facility for reading the SmartCard technology.

### BRIEF DESCRIPTION OF THE DRAWINGS

A more complete understanding of the invention may be had by reference to the accompanying Drawings, wherein:

FIG. 1 is a perspective view illustrating an apparatus for acquiring, storing, and transmitting patient medical data comprising the preferred embodiment of a invention; and

FIG. 2 is an illustration of the reverse side of the apparatus of FIG. 1.

### DETAILED DESCRIPTION

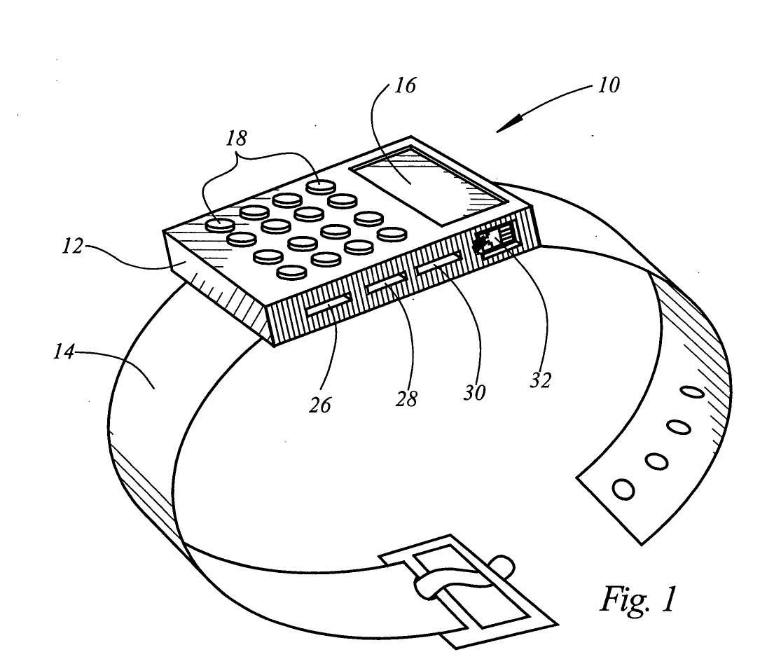

Referring to the Drawings, and particularly to FIG. 1 thereof, there is shown an apparatus for acquiring, storing, and transmitting patient medical data 10 comprising the preferred embodiment of the invention. The apparatus 10 includes a case or housing 12 supported on a strap 14 and is therefore adapted to be secured to the wrist of the patient. The apparatus 10 is provided with a conventional miniature display 16 and a conventional miniature keyboard 18 which facilitates the entry of data into the apparatus 10. Voice recognition software can also be used to effect input to the apparatus 10.

An important feature of the present invention comprises a memory which is contained within the case 12 of the apparatus 10. Preferably, the memory of the apparatus 10 is sufficient capacity to store all of the medical records of the patient. Thus, the means of the memory of the apparatus 10 all of the medical records of the patient are carried by the patient, rather than being stored at a doctor's office or other remote location. By this means there is provided access to the entire medical history of the patient at any time and at any location at which the patient may require medical assistance.

The apparatus 10 further includes circuitry for inputting data to and recovering data from the memory. Such data includes e-mail and telephone number addresses for locations to which patient medical data is to be transmitted. Such locations include the office of the doctor that is attending the patient, a laboratory associated with the doctor's office, a patient medical data search facility such as Medic Alert (TM), and possibly data storage facilities at the home or office of the patient.

In case of an emergency, the apparatus 10 can also transit the medical history of and current medical data pertaining to the patient to a 911 facility, a hospital emergency room, etc. To further facilitate emergency medical treatment for the patient, the apparatus 10 includes a GPS receptor. The current location of the patient as determined by the GPS system is continuously stored within the memory of the apparatus 10 and is therefore immediately available for transmission to emergency personnel so that the patient can be located and treated substantially instantly.

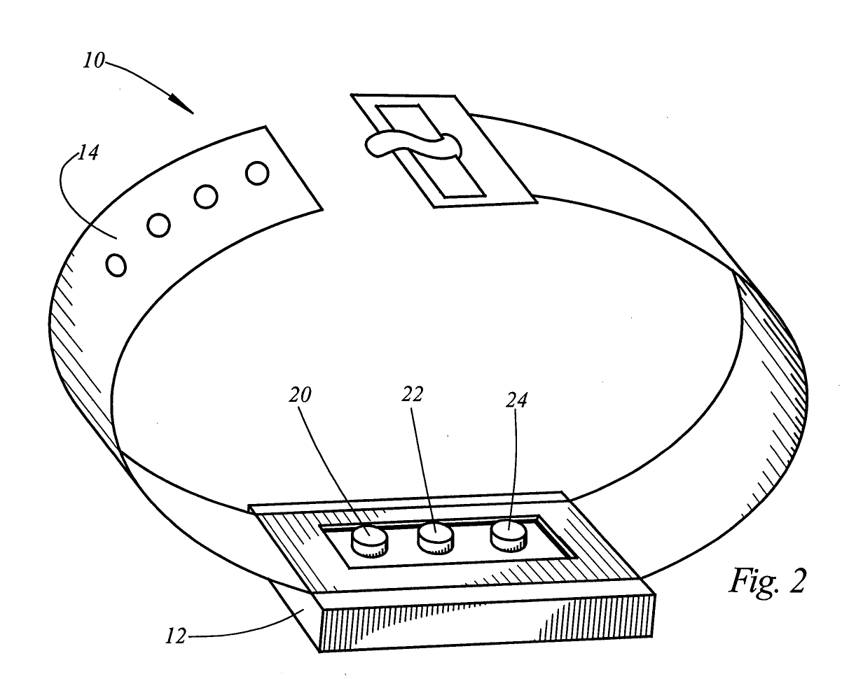

Referring to FIGS. 1 and 2, the apparatus 10 is provided with one or more sensors 20, 22, and 24 which directly acquire medical data such as temperature, pulse, blood pressure, etc. The apparatus 10 is also provided with one or more slots 26, 28, and 30 which receive strips each bearing a droplet of the blood of the patient. In the apparatus 10 there is provided circuitry for analyzing the blood of the patient when a strip bearing the blood is inserted into the appropriate slot. In this manner there is acquired data such a cholesterol levels, blood sugar level, blood thinner level, etc. for storage in the memory.

Within the apparatus 10 there is provided a modem and circuitry for retrieving from the memory address information such as telephone numbers, e-mail addresses, etc. The modem within the apparatus 10 is connected to communications facilities through a conventional telephone jack 32. Conventional wireless connections, such as infrared connection devices, can also be used.

In the operation 7 of the present invention, the apparatus 10 is secured on the wrist, leg, etc., of the patient using the strap 12. In the case of a healthy patient, it is not necessary to wear the apparatus 10 constantly but only occasionally when the acquisition, storage, and transmission of medical data is indicated. In the case of a patient having significant disease, it may be prudent to wear the apparatus 10 on a continuous basis in order that patient medical data can be acquired, stored, and transmitted whenever is needed.

GPS data is constantly acquired by the apparatus 10 so that the apparatus 10 continuously contains information as to the whereabouts of the patient. Addresses for the locations to which patient medical data is to be sent are inputted, updated, and selected using the keyboard 18 in conjunction with the display 16. The display 16 may be also be used to provide the patient with an instantaneous readout of acquired medical data.

Medical data acquired by the apparatus 10 is immediately stored in the memory thereof. Thus, the medical history of the patient which is stored in the memory of the apparatus 10 is updated on every occasion when medical data is acquired from the patient utilizing the apparatus 10. Following acquisition of the medical data, the telephone jack 32 of the apparatus 10 is utilized to connect the apparatus 10 to a desired location either utilizing a conventional telephone connection, or via the Internet using e-mail. Other forms of data transmission may be utilized in the practice of the invention, if desired.

Although preferred embodiments of the invention have been illustrated in the accompanying Drawings and described in the foregoing Detailed Description, it will be understood that the invention is not limited to the embodiments disclosed, but is capable of numerous rearrangements, modifications, and substitutions of parts and elements without departing from the spirit of the invention.

1. An apparatus for acquiring, storing, and transmitting patient medical data including:
a housing securable on the body of a patient;
a memory mounted within the housing for storing patient medical data;
circuitry mounted within the housing for inputting patient medical data and other data to and for retrieving patient medical data and other data from the memory;
a keyboard mounted on the housing for inputting data to and retrieving data from the memory;
a display mounted on the housing for displaying data inputted to and retrieved from the memory;
at least one sensor mounted in the housing for contact with the skin of a patient to acquire patient medical data;
a GPS detector for inputting patient location data to the memory;
at least one slot extending into the housing for receiving blood-bearing strips and thereby acquiring patient medical data;
a modem mounted in the housing for receiving patient medical data and GPS data from the memory; and
connection apparatus mounted in the housing for connecting the modem to a preselected communication system.
